(19) United States
(12) Patent Application Publication
(10) Pub. No.: US 2005/0063938 A1
(43) Pub. Date: Mar. 24, 2005

(54) Tertiary amine functional complex polyester polymers and methods of production and use

(76) Inventors: Rocco Vincent Burgo, Mullica Hill, NJ (US); Diana Louise Smith, Tabernacle, NJ (US); Hor P. Ching, Upper Darby, PA (US)

Correspondence Address: WOLF, BLOCK, SCHORR & SOLIS-COHEN LLP, 1650 ARCH STREET, 22ND FLOOR, PHILADELPHIA, PA, 19103-2334, US

(21) Appl. No.: 10/779,306

(22) Filed: Feb. 13, 2004

Related U.S. Application Data
(60) Provisional application No. 60/447,530, filed on Feb. 14, 2003.

(51) Int. Cl. A61K 31/785

(52) U.S. Cl. 424/78.27

(57) ABSTRACT

A polyesteramine comprises at least one tertiary amine group, at least one ester linkage and at least one alkyl chain. It is produced by reacting at least one tertiary amine functional polyol, at least one polyfunctional carboxylic acid and at least one monofunctional carboxylic acid or monofunctional alcohol. The polyesteramine can be used in cosmetic and lubricant applications as a result of its substantivity and lubricity properties.

### CROSS-REFERENCE TO RELATED APPLICATION

This application claims the benefit of U.S. Provisional Application No. 60/447,530, filed Feb. 14, 2003, the entire disclosure of which is incorporated herein by reference.

### FIELD OF THE INVENTION

This invention relates to improved additives for cosmetic and lubricant applications comprising tertiary amine functional complex polyester polymers (“polyesteramines”) derived from polycarboxylic acids, tertiary amine functional polyols, monofunctional carboxylic acids and/or monofunctional alcohols, and optionally polyols and/or hydroxyacids, their method of production, and their use.

### BACKGROUND OF THE INVENTION

A broad variety of ingredients have been used for hair and skin conditioning formulations and additives. Since keratinous substances, especially the hair, are normally negatively charged because of their low isoelectric point, conditioning additives normally contain one or more cationic sites that bind the remainder of the molecule to the skin and/or hair by electrostatic attraction. Other portions of the molecule typically consist of long chain, hydrophobic alkyl groups that serve to provide a lubricious film on the substrate, or in the case of polymeric types, repeating units of linear and/or branched alkyl groups. These ingredients are useful in personal care products for application to the hair such as conditioners, conditioning shampoos, mousses, gels, sprays, waxes, and other styling aids. They are also useful in personal care products for application to the skin such as moisturizing creams and lotions, body washes, bath gels, shaving creams, shaving gels, and liquid body detergents.

Both non-polymeric cationic and polymeric cationic materials are used extensively for these applications, and normally consist of one or more cationic moieties and alkyl portions. Cationic moieties are typically either quaternized nitrogen atoms or neutralized amine groups. The alkyl portions are normally derived from fatty acids obtained from the hydrolysis of oils and/or fats derived from vegetable and/or animal sources, or from chemical synthesis from petrochemically derived starting materials.

One class of common non-polymeric conditioning additives are alkamidopropyldimethylamines that are derived from the amidization of long chain fatty acids typically containing from 12 to 24 carbon atoms with dimethylaminopropylamine. Typical of these are stearamidopropyldimethylamine and behenamidopropyldimethylamine (Lexamine® S-13 and Lexamine® B-13, Inolex Chemical Company, Philadelphia, Pa., USA). These compounds are typically solid materials with melting points above 65° C., are supplied in flake form, and are therefore difficult to handle and process. Furthermore, due to their low molecular weight (less than 450 Daltons), they tend to be irritating to the eyes and the skin.

A second class of common non-polymeric conditioning additives are alkyldimethylamine quaternaries such as cetrimonium chloride (Barquat CT-29, Lonza Incorporated, Annandale, N.J., USA) and behentrimonium chloride (Incroquat Behenyl TMC, Croda Corporation, Parsippany, N.J., USA). In their pure state, alkyldimethylamine quaternaries are very high melting solids and, like the alkylamidoamines are difficult to work with in their pure, 100% active state. They too are relatively low in molecular weight, less than 410 Daltons, and are also irritating to the skin and eyes.

A third class of non-polymeric conditioning additives are ester quaternaries. They are typically derived by the full or partial esterification of a trialkanolamine, typically triethanolamine, followed by the quaternatization of the tertiary nitrogen atom with methyl chloride or dimethyl sulfate. Although originally developed as improved laundry detergent surfactants due to their enhanced biodegradability, they have later found use in personal care applications as conditioning additives. The enhanced biodegradability comes from the fact that they contain ester linkages that can hydrolyze over time, especially when in contact with esterase enzymes normally found in wastewater treatment plants. Commercial examples of these are dicocoylethyl hydroxyethylmonium methosulfate and dehydrogenated tallow hydroxyethylmonium methosulfate (Dehyquart® L-80 and Dehyquart® AU-56, respectively, Cognis Corporation, Hoboken, N.J., USA.). Ester quaternaries are also high melting solids or pastes which makes them difficult to use in their 100% active state. To overcome this, they are sometimes supplied as blends with co-emulsifiers or other solvents which sometimes force the formulator to inadvertently add these other components into the final formulation.

Although nonionic polymers are sometimes used in hair and skin conditioning applications, in general, most of the currently used polymeric conditioning additives are cationic polymers in which the cationic binding sites are provided by quaternization of nitrogen atoms within the backbone of the molecule. Some commonly used examples are quaternized poly(vinylpyrrolidone/dimethylaminoethyl methacrylate) (Gafquat 744, ISP Corporation, Wayne, N.J., USA) and N,N-dimethyl-N-2-propen-1-aminium chloride homopolymer (Merquat 100, ONDEO Nalco Corporation, Naperville, Ill., USA). Due to the relatively high molecular weight of these compounds, they tend to exhibit lower irritancy than alkamidodimethylamines and alkyldimethylamine quaternaries. However, polymers of this type tend not to be biodegradable due to the absence of ester linkages. They are also very high melting solids that are sparingly soluble in water, and thus can be supplied as only very dilute solutions. Furthermore, due to the very sophisticated processes required for their production, they are very costly. Lastly, cationic polymers of this type, due to their relatively high charge density, tend to build up on the skin and hair and can tend to provided negative sensory feeling upon continued use.

Thus, in cosmetic applications, it is difficult to use certain typical conditioning additives because they are solid materials and/or difficult to use products, and/or are irritating to the skin and/or eyes, and/or are poorly biodegradable, and/or are too costly. In addition, there is always a need to improve the effectiveness of these ingredients in their ability to provide conditioning benefits. More effective conditioning ingredients enable the creation of products of superior performance. Additionally, ingredients with improved properties allow for the reduction of the amount of additive used, and this, in conjunction with lower additive cost, can contribute to a higher cost effectiveness to the cosmetic manufacturer.

In the field of lubricant additives, especially in applications wherein the lubricant is water dilutable such as in metalworking cutting fluids and coolants, polyol polyester polymers are used as both anti-wear (AW) and extreme pressure (EP) additives. These materials are being selected more extensively due to the heightened awareness over the potential toxicities associated with traditionally used EP additives such as chlorinated paraffins. For example, non-ethoxylated polyol polyester polymers with low residual carboxylic acid content are currently used as lubricity additives that provide both AW and EP behavior. Polyol polyester polymers of this type are effective lubricants and generally exhibit a low tendency to irritate the skin and/or the eyes, and are waste treatable. Since the actual composition of these materials are trade secrets and only their general composition is disclosed, provided examples of commercial products are only generically described. Examples are Lexolube® CQ 3000 (Inolex Chemical Company, Philadelphia, Pa., USA) and Syn-Ester GY-25L (The Lubrizol Corporation, Wickliffe, Ohio, USA). However, due to their absence of strongly hydrophilic groups, materials of this type can be difficult to emulsify.

Commercial polyol polyester polymer lubricant additives are available that claim to be “self-emulsifying” and/or “easily emulsifiable.” Typical of these are polyol polyester polymers containing a significant level of residual carboxylic acid functionality and/or ethoxylation. A commercial example of a polyol ester polymer containing higher residual carboxylic acid functionality to improve emulsifiability is Syn-Ester GY-25. When formulated into typical water dilutable metalworking fluids which are typically buffered to a pH of about 8.5 to about 9.5, residual carboxylic acid groups are neutralized to their respective salts which contributes to easier emulsifiability. However, this also contributes to higher levels of foam, hard water scum, the formation of biofilms, and other conditions that result in a general lack of cleanliness. Inclusion of moities that result from ethoxylation will tend to increase the level of foam even further. A commercial example of a polyol polyester polymer that contains both carboxylic acid groups and ethoxylation is Priolube 3952 (Uniqema, Wilmington, Del., USA). Poor emulsifiability, hard water scum, the formation of biofilms, lack of cleanliness, and foam are all significant disadvantages in metalworking lubricant applications.

Thus, there is a need for additives to be used in cosmetic and lubricant applications that improve on the properties of previously used additives.

### SUMMARY OF THE INVENTION

The present invention comprises polyesteramine compounds that result when polycarboxylic acids, tertiary amine functional polyols, monofunctional carboxylic acids and/or monofunctional alcohols, and, optionally, polyols and/or hydroxyacids are esterified. The polyesteramines have a low melting point, are easy to use and are 100% active products. They are used both in cosmetics and in lubricants. Furthermore, since they are polymeric, they are non-irritating to the skin and eyes.

The polyesteramines of the present invention have multiple tertiary amine sites per molecule that when neutralized to the cationic form provide greater substantivity to skin and hair, and are better conditioners in typical personal care applications. Additionally, in their unneutralized state they easily emulsify in water due to the polar tertiary amine groups and exhibit “self emulsifying” behavior without the use of significant ethoxylation or significant carboxylic acid termination. Additionally, the inventive polyesteramines provide excellent lubricity in lubrication applications. Because the inventive polyesteramines contain ester linkages, they have a good biodegradability profile.

### BRIEF DESCRIPTION OF FIGURES

FIG. 1 shows a mildly crosslinked polyesteramine derived from fatty acid, MDEA, glycerol, and adipic acid.

FIG. 2 shows a linear polyesteramine derived from fatty acid, MDEA, and adipic acid.

FIG. 3 shows a crosslinked polyesteramine derived from fatty acid, MDEA, glycerol, and adipic acid.

FIG. 4 is a diagram depicting the mode of action of the inventive polyestermine compound.

### DETAILED DESCRIPTION

The present invention comprises polyesteramines, defined above as complex tertiary amine functional polyester polymers and described below in more detail. The polyesteramines are used in both cosmetic and lubricant applications. The polyesteramines are low melting viscous liquids with melting points ranging from about −40° C. to about 35° C., and viscosity ranging from about 1,000 centipoise to about 10,000 centipoise when measured at 25° C. The preferred molecular weight range for the polyesteramines is between about 600 and about 5,000 Daltons. A preferred acid value is from 0 to about 100 mg KOH/g for cosmetics, and from 0 to about 20 mg KOH/g for lubricant applications. A preferred amine value is from about 20 to about 200 mg KOH/g.

One method of producing a polyesteramine of the invention comprises co-esterifying at least one tertiary amine functional polyol, at least one polyfunctional carboxylic acid, and at least one monofunctional carboxylic acid and/or alcohol, and optionally, a polyol and/or a hydroxyacid. The ratio of reactants, the type of reactants, and the reactant properties can be varied to control the physical form and properties of the polyesteramines, e.g., viscosity, solubility, emulsifiability, substantivity and lubricity. Chemical compounds having similar properties to the above-recited reactants may be substituted therefor.

A preferred tertiary amine functional reactant is methyldiethanolamine (MDEA). Preferred polyfunctional carboxylic acids are adipic acid, cyclohexanedicarboxylic acid, sebacic acid, azelaic acid, dodecanedioic acid, phthalic acid, isophthalic acid, terephthalic acid, trimellitic acid, dimer acid, trimer acid, 2,6-naphthalene dicarboxylic acid, and pyromellitic acid. Preferred monofunctional carboxylic acids are benzoic acid, 2-ethylhexanoic acid, isononanoic acid, lauric acid (C-12), myristic acid (C-14), palmitic acid (C-16), isomyristic acid (Iso C-14), isopalmitic acid (Iso C-16), isostearic acid (Iso C-18), coconut fatty acid (C8-C18), oleic acid (C18:1), and behenic acid (C-22). Preferred monofunctional alcohols are tridecyl alcohol, Guerbet alcohols, coconut fatty alcohols, isooleic alcohol, and isostearyl alcohol. Preferred polyols are propylene glycol, 1,3-butylene glycol, cyclohexanedimethanol, trimethylpentanediol, polyoxyalkylene glycol, butyl ethyl propanediol, dipropylene glycol, neopentyl glycol, glycerol, trimethylolpropane, pentaerythritol, and dipentaerythritol. Preferred hydroxy acids are lactic acid, glycolic acid, hydroxystearic acid, and citric acid.

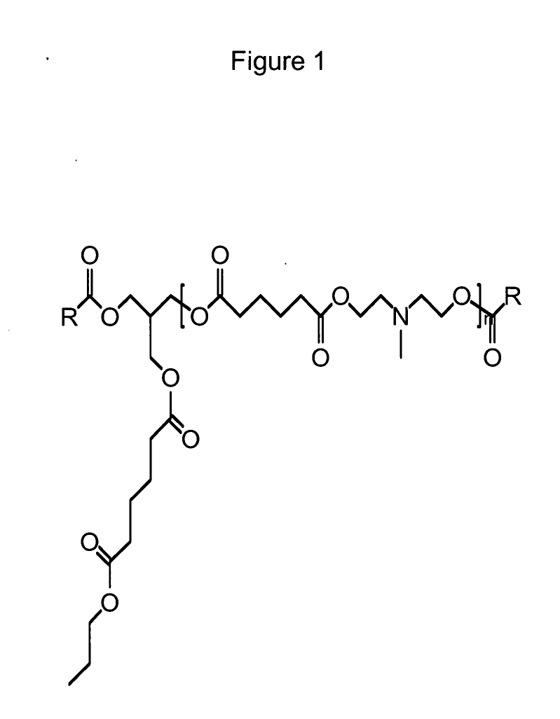

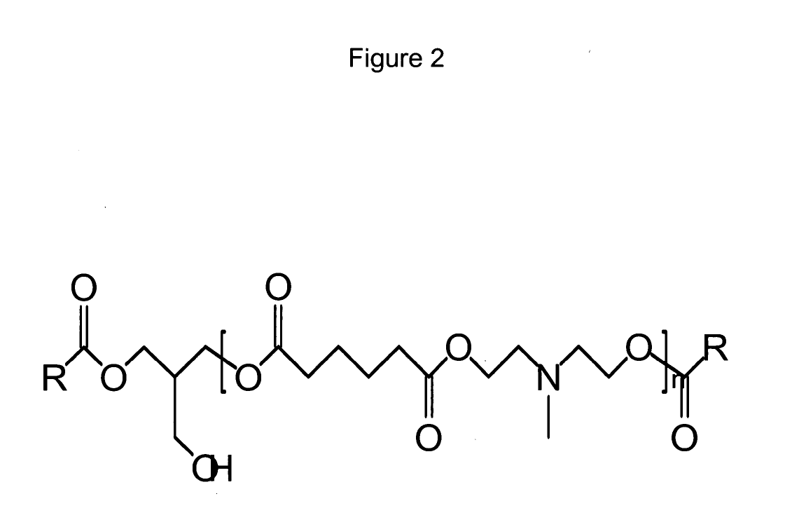

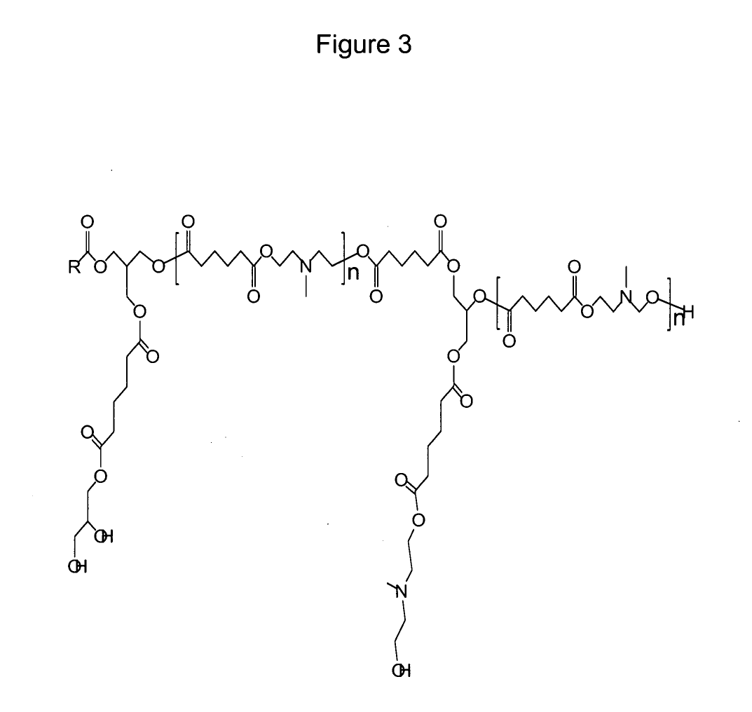

The reactants described above are combined to yield polymeric molecules. Exemplary structures are shown in FIGS. 1 to 3. By combining the reactants in different molar ratios, the molecular weight, crosslink density, alkyl chain density, and tertiary amine density are controlled to produce properties desired for a particular application. By varying these chemical attributes, physical attributes, such as physical form, viscosity and solubility, and applicational properties, such as substantivity, emulsifiability, and lubricity, can be controlled.

In FIGS. 1-3, R is a carbon chain of from about 5 to about 35 carbon atoms derived from vegetable oils, animal fats and/or oils, or from chemical synthesis. The carbon chain may be unbranched or branched and saturated or unsaturated and non-aromatic or aromatic.

The polyesteramine technology of the invention is designed such that the polymeric molecules contain tertiary amine groups, ester linkages, alkyl chains, and, optionally, hydroxyl groups, and/or carboxylic acid groups.

The substrate (i.e., surface to which they are applied) in cosmetic applications is either the hair or the skin. Both the hair and skin are negatively charged, thus, the most effective conditioning agents are cationic. Cationic conditioning agents act by binding a moiety, typically a long chain alkyl group, to the hair or skin by electrostatic attraction (ionic bonding), but also, weaker forces such as dipole-dipole interactions, and furthermore van der Waals forces can assist in substrate binding. Substantivity is a term used to describe how well a conditioning agent binds to the surface of the substrate. Substantivity of hair conditioning agents is often determined by performing the Rubine Dye test. The Rubine Dye test is designed to show how much of the conditioning ingredient adheres to the hair. Results from the Rubine Dye test are normally evaluated by comparison to some known standard or benchmark.

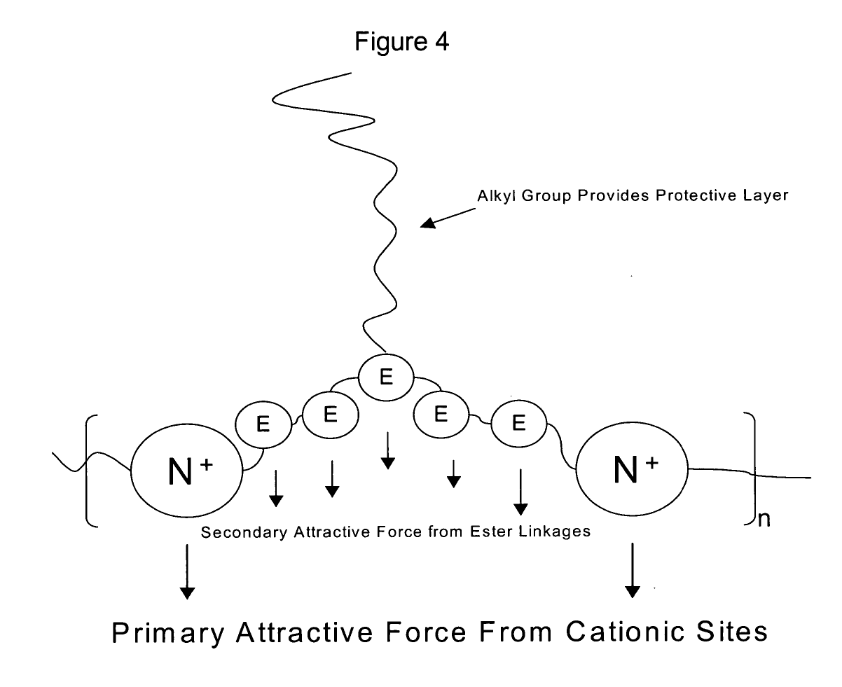

As previously described, some more common cationic conditioning agents are non-polymeric molecules consisting of a single cationic site, and a long chain alkyl group. Increasing the number and type of binding sites in the molecule will increase the substantivity. Additionally, increasing the number of alkyl chains per molecule results in the formation of a thicker, more lubricious film providing the conditioning effect. FIG. 4 depicts the mode of action for the polyesteramine technology in hair and skin care applications. In FIG. 4, the alkyl group is shown to provide a protective layer, and also shown are the primary attractive forces from cationic linkages (ionic bonding) and secondary attractive forces from ester linkages (dipole-dipole interactions.)

By controlling the degree of polymerization (n), molecules can be made with multiple primary and secondary sites of attractive force to the substrate, as well as multiple alkyl groups per molecule that upon entanglement, provide the lubricious film. In this way, the properties of the resulting polyesteramine can be controlled.

Additionally, because the cationic nature of the polyesteramine is provided by tertiary amino groups and not quaternary ammonium groups, as in the case of alkyltrimonium chlorides, ester quaternaries, and polyquaternaries, the strength of attachment to the substrate can be controlled by controlling the pH of the formulation. At low pH (between about 4.0 and about 5.0), which is typical for hair conditioner formulations, essentially all of the tertiary amino groups are protonated, and are thus in the cationic state. At intermediate pH (between about 5.0 and about 8.0), less of the amino groups are in the cationic state. The pH control provides tremendous flexibility to the formulator of hair and skin conditioning products since the protonated molecules can provide conditioning, while the unprotonated molecules provide primary and/or secondary emulsification. Additionally, by controlling the pH of the formulation, the intensity of the conditioning effect and/or the tendency for the conditioning agent to build-up on the hair can be controlled. Furthermore, the ester groups in the backbone structure of polyesteramines provide secondary attractive force between the conditioning molecule and the substrate. Additionally, since polyesteramine chemistry is polymeric, using the method described, the resulting products are mixtures (with molecular weight distributions). By varying the average molecular weight, the strength of the conditioning effect may be controlled.

In lubricant applications, the substrate is typically a ferrous or non-ferrous metal alloy. Metals typically contain an oxide layer at the surface. As discussed previously, polyesteramines according to the invention contain ester groups, tertiary amine groups, and alkyl chains. Since the ester linkage is polar, the ester groups will tend to bind by adsorption to the oxide layer on metal surfaces. The alkyl chains do not bind and provide a lubricious film. The highly polar hydrophilic tertiary amine moieties within the molecule make it easy to disperse and emulsify the polyesteramine lubricant into water, which is a benefit in water-based applications, such as water dilutable metalworking fluids.

### EXAMPLE 1

When evaluating the invention, an initial matrix of prototypes were developed to evaluate their properties. Each experimental polyesteramine prototype is identified by Types 1-6. Table 1 below shows the number of moles of each ingredient employed for each prototype. By varying properties, such as molecular weight, alkyl chain type and density, polyol type and density, and tertiary amine group density, the structure and performance of each prototype can be controlled.

The prototypes were prepared by charging the ingredients to a stirred batch reactor in the presence of a small quantity of antioxidant to preserve color. The reactants were heated with continuous inert gas sparging to between about 170° C. and about 200° C. The acid value and amine value were monitored, and the reaction was stopped by cooling when the acid value reached 10 or lower.

TABLE 1
Summary of Properties of Polyesteramine Prototypes Type 1 through Type 6.

| | Type 1 | Type 2 | Type 3 | Type 4 | Type 5 | Type 6 |
|---|---|---|---|---|---|---|
| Component | | | | | | |
| Moles MDEA | 8.7 | 9.1 | 6.3 | 4.4 | 3.5 | 3.2 |
| Moles Adipic | 4.1 | 5.9 | 4.5 | 4.1 | 5.9 | 4.8 |
| Moles Polyol | 0.8 | 0.7 | 3.0 | 3.1 | 4.2 | 3.2 |
| Moles Acid | 3.1 | 2.0 | 1.5 | 3.1 | 2.5 | 3.2 |
| Polyol Moiety | Prop Glycol | Glycerol | Glycerol | Glycerol | Glycerol | Glycerol |
| Alkyl Moiety | Palmityl | Palmityl | Palmityl | Palmityl | Palmityl | Isostearyl |
| Property | | | | | | |
| MW (Daltons) | 450 | 600 | 700 | 800 | 1200 | 1300 |
| Crosslink | 0.0 | 0.2 | 0.5 | 1.0 | 2.3 | 2.0 |
| Density | | | | | | |
| 3° Amine | 1.5 | 2.5 | 2.0 | 1.0 | 2.0 | 2.0 |
| Density | | | | | | |
| Fatty Alkyl | 0.5 | 0.5 | 1.0 | 1.0 | 1.5 | 2.0 |
| Density | | | | | | |
| Acid Value | 6.2 | 5.6 | 2.3 | 5.6 | 3.8 | 8.0 |
| (mg KOH/g) | | | | | | |
| Amine Value | 235 | 225 | 161 | 123 | 95 | 92 |
| (mg KOH/g | | | | | | |
| Equiv. Wt. | 239 | 249 | 351 | 456 | 591 | 610 |
| (Daltons) | | | | | | |
| Physical Form | Liquid | Liquid | Liquid | Solid | Solid | Liquid |

### EXAMPLE 2

The polyesteramine prototypes were tested internally for substantivity using the Rubine Dye test protocol. In the Rubine Dye test, the prototypes were benchmarked against Dehyquart® L-80 (dicocoylethyl hydroxyethylmonium methosulfate (and) propylene glycol), a commercial ester quaternary. The materials and equipment used in performing the Rubine Dye analysis were:
Bleach blond hair (De Meo Brothers);
Lumicrease Bordeaux 3LR powder-Dye (Clariant Corporation);
Glacial acetic acid, UPS grade;
Hydrogen peroxide, 10% solution;
Glue gun;
Glue sticks;
Plastic sheet;
Chromameter (Minolta CR-300);
Digital camera (Olympus digital camera, model C2020Z);
Polyesteramine Type 1;
Polyesteramine Type 2;
Polyesteramine Type 3;
Polyesteramine Type 4;
Polyesteramine Type 5;
Polyesteramine Type 6; and
Dehyquart L-80 (Cognis Corporation).

The protocol used to perform the Rubine Dye analysis on polyesteramine prototypes and Dehyquart® L-80 and the compositions of ingredients used is described below.

First, a stock dye solution was prepared using the following formula and preparation procedure:

| Component | Parts By Weight |
|---|---|
| Deionized water | 99.37 |
| Lumicrease Bordeaux 3LR Powder | 0.50 |
| Glacial acetic acid | 0.13 |

In a small vessel, deionized water, Lumicrease Bordeaux 3LR Powder, and glacial acetic acid were agitated at about 20 to about 25° C. until a solution was obtained.

Second, a test dye solution was prepared using the following formula and mixing procedure:

| Component | Parts By Weight |
|---|---|
| Deionized water | 80.00 |
| Stock Dye Solution | 20.00 |

In a small vessel, deionized water and stock dye solution were agitated at about 20° C. to about 25° C. until a solution was obtained.

Third, a separate conditioner base formula for each of the prototypes to be tested was prepared using the following formula and formulation procedure:

| | |
|---|---|
| Component | Parts By Weight |

Part A

| | |
|---|---|
| Deionized water | QS to 100% |
| Tetrasodium EDTA | 0.05 |
| Methylparaben | 0.20 |
| Propylparaben | 0.15 |
| Polyesteramine/quat | 2.00 |

Part B

| | |
|---|---|
| Glyceryl Stearate (and) PEG-100 Stearate | 4.00 |
| Cetyl alcohol | 3.00 |
| Mineral Oil | 2.00 |

Part C

| | |
|---|---|
| Hydrochloric Acid (31%) | QS to pH 5.0-5.2 |

In a small vessel with agitation, deionized water, methylparaben, propylparaben, and the conditioning agent to be tested (polyesteramine prototype or benchmark product) were combined and warmed to about 75° C. to about 80° C. In a separate vessel, mineral oil, cetyl alcohol, and glyceryl stearate (and) PEG-100 stearate were combined with agitation and warmed to about 75° C. to about 80° C. The contents of one vessel was poured into the other, and the mixture was allowed to cool to about 20° C. to about 25° C. and an emulsion was formed. At that point, the pH of the emulsion was greater than about 5.0 to about 5.2, and was then adusted downward to this range with hydrochloric acid solution. The entire procedure was also performed without any conditioning ingredient so as to act as a control.

From about 0.95 to about 1.05 grams of hair was weighed out and glued onto pre-cut plastic sheets (1.5″×1.5″) using a hot glue gun to attach the swatches to the sheets. It was repeated as necessary to make enough hair swatches for the study. The hair swatches were then bleached in a 10% hydrogen peroxide solution for about ten minutes, rinsed for about six minutes, and air-dried. The hair swatches were individually rinsed under warm (40° C.) running tap water. 2.00 grams of cationic conditioner was massaged onto each hair swatch and allowed to remain on the swatch for one minute. Each hair swatch was then rinsed under running water for two minutes and excess water was removed by blotting with a paper towel. Each hair swatch was then immersed in 200 mililiters of test dye solution for ten seconds then rinsed under running water for five seconds before being patted between paper towels to remove excess water, and then air dried. Quantitative comparisons of dye up-take and relative substantivity of each polymer were performed using a Minolta chromameter. Additionally, dye up-take was evaluated qualitatively by taking photographs of the hair swatches using a digital camera.

The Rubine Dye analysis is used in the personal care industries to evaluate the substantivity of a molecule onto hair. The more dye deposits on hair the redder the hair swatches get. The Minolta chromameter measures the intensity of the color of the hair swatches as an absolute color based upon the tristimulus analysis of a reflected Xenon light pulse. The result is expressed as a three dimensional coordinate consisting of two color coordinates (the green-red or a-scale and the yellow-blue or b-scale) and a luminescence coordinate (black-white or L-scale). These three coordinates (a, b, and L) define the absolute color of the hair swatches, with higher values on the a-scale and lower values on the L-scale and b-scale relating to higher amounts of deposition of the conditioning ingredient. Table 2 shows the substantivity of each tested product. Each value listed in the table is the average of three separate measurements.

TABLE 2
Chromameter data on polyesteramine prototypes
as benchmarked against Dehyquart L-80.

| Test Material | L-scale value | a-scale value | b-scale value |
|---|---|---|---|
| Control | 78.93 | 3.30 | 20.40 |
| Type 1 | 76.07 | 8.41 | 16.94 |
| Type 2 | 76.57 | 7.05 | 16.96 |
| Type 3 | 74.05 | 10.69 | 15.27 |
| Type 4 | 71.06 | 14.45 | 12.33 |
| Type 5 | 63.64 | 23.90 | 6.77 |
| Type 6 | 57.79 | 29.59 | 5.25 |
| Dehyquart ® L-80 | 68.01 | 16.70 | 9.16 |

The results show that polyesteramine backbone prototype Type 6 was the most substantive molecule, and both polyesteramine Type 5 and Type 6 were significantly more substantive than Dehyquart® L-80. After obtaining the results, these prototypes were evaluated applicationally. Their evaluation showed that polyesteramine Type 5 and polyesteramine Type 6 performed better than the other prototypes.

### EXAMPLE 3

A second matrix of polyesteramine products was then designed and synthesized to further explore the structure/performance relationships with molecules surrounding the structural characteristics of the better performing polyesteramines, the Type 5 and Type 6 products. The synthesis method previously described was again used. Table 3 shows the results obtained from these prototypes. The above materials were also evaluated using the Rubine Dye test for substantivity. Table 4 lists the results obtained.

TABLE 3
Summary of properties of improved polyesteramine prototypes.

| | S 1500 | S 1750 | O 1250 | O 1500 | O 1700 | C 1150 | C 1450 | C 1600 |
|---|---|---|---|---|---|---|---|---|
| Component | | | | | | | | |
| Moles MDEA | 3.0 | 4.0 | 2.0 | 3.0 | 4.0 | 2.0 | 3.0 | 4.0 |
| Moles Adipic | 4.0 | 5.0 | 3.0 | 4.0 | 5.0 | 3.0 | 4.0 | 5.0 |
| Moles Polyol | 2.0 | 2.0 | 2.0 | 2.0 | 2.0 | 2.0 | 2.0 | 2.0 |
| Moles Acid | 2.0 | 2.0 | 2.0 | 2.0 | 2.0 | 2.0 | 2.0 | 2.0 |
| Polyol Moiety | Glycerol | Glycerol | Glycerol | Glycerol | Glycerol | Glycerol | Glycerol | Glycerol |
| Alkyl Moiety | Iso C18 | Iso C18 | C-18:1 | C-18:1 | C-18:1 | C8-C18 | C8-C18 | C8-C18 |
| | | | (oleic) | (oleic) | (oleic) | (coco) | (coco) | (coco) |
| Property | | | | | | | | |
| Physical State | Liquid | Liquid | Liquid | Liquid | Liquid | Liquid | Liquid | Liquid |
| Color, Gardner | 1+ | 1+ | 2+ | 4+ | 2+ | 0+ | 0+ | 0+ |
| Odor, (olfactory) | Amine | Amine | Slightly | Slightly | Slightly | Very | Very | Very |
| | | | Rancid | Rancid | Rancid | Mild | Mild | Mild |
| Acid Value | 4.67 | 2.76 | 4.56 | 8.72 | 8.44 | 6.20 | 3.80 | 5.87 |
| (mg KOH/g) | | | | | | | | |
| Amine Val. | 109 | 122 | 81.6 | 109 | 124 | 93.9 | 111 | 133 |
| (mg KOH/g) | | | | | | | | |
| Equiv. Wt. | 515 | 460 | 688 | 515 | 452 | 598 | 507 | 422 |
| (Daltons) | | | | | | | | |
| Visc.@25 C. | 3480 | 4360 | 990 | 2440 | 2725 | 2270 | 4805 | 4505 |
| (cps) | | | | | | | | |

TABLE 4
Results of Substantivity Testing On Prototypes.

| Test Material | L-scale value | a-scale value | b-scale value |
|---|---|---|---|
| S 1500 | 49.94 | 31.59 | 2.27 |
| S 1750 | 52.25 | 30.00 | 3.19 |
| O 1250 | 50.84 | 33.34 | 5.10 |
| O 1500 | 48.89 | 32.30 | 4.16 |
| O 1700 | 48.94 | 31.55 | 2.56 |
| C 1150 | 44.31 | 30.17 | 0.90 |
| C 1450 | 42.38 | 30.85 | 2.04 |
| C 1600 | 48.69 | 32.07 | 2.27 |
| Type 6 | 50.06 | 29.15 | 3.51 |
| Lexamine ® S-13 | 65.15 | 19.16 | 8.57 |

The results on the Rubine Dye test for the improved polyesteramine prototypes indicated that virtually all of the polyesteramines were more substantive than the commercial conditioning additive Lexamine® S-13, and that polyesteramine prototypes C 1450 and Type 6 were the most substantive.

### EXAMPLE 4

Irritancy to the eyes and to the skin was tested in-vitro using the EpiDerm® and EpiOccular® test models. EpiDerm® is a human test model of skin made up of normal epidermal keratinocytes. The EpiOccular® model is made of human-derived epidermal keratinocytes cultured to make them similar to human corneal tissue. In each of the tests, tissue samples are treated with the test article for various exposure times. Following treatment, the viability of the tissue is determined using MTT uptake and conversion, and the absorbance of the sample is measured at 540 nanometers (nm) wavelength using a reference wavelength of 690 nm. The viability is expressed as a percentage of control values. The mean percentage viability for each time point is used to calculate the ET₅₀, which represents the time at which the tissue viability is reduced 50% compared to control tissue. Polyesteramines Type 6 and C 1150 were evaluated using the EpiOccular® model for exposure times of 16, 64, and 256 minutes, and the EpiDerm® model for exposure times of 1, 4, and 24 hours. These prototypes were chosen because Type 6 was very high performing in the substantivity test, and C 1150 was the lowest in molecular weight of the improved prototypes. Typically, higher molecular weight results in lower eye and skin irritancy. Table 5 shows the results obtained:

TABLE 5
ET₅₀ results for polyesteramines Type 6 and C 1150 using
the Epiderm ® and EpiOccular ® test models.

| | | EpiDerm ® |
|---|---|---|
| Prototype | EpiOcular ® ET₅₀ (min.) | ET₅₀ (hrs.) |
| Polyesteramine Type 6 | >256.0 | >24 |
| Polyesteramine C 1150 | >256.0 | >24 |

ET₅₀ of greater than 256 minutes on the EpiOcular® model and greater than 24 hours on the EpiDerm® model corresponds to irritancy classifications of non-irritating since the end point of tissue viability being reduced to 50% is never obtained over the duration of the tests. Each of the polyesteramine prototypes Type 6 and C 1150 are classified as non-irritating to the eyes and skin. For comparative purposes, cetrimonium chloride has an ET₅₀ of 116.9 minutes using the EpiOcular® test model which indicates that it is more irritating.

The formulations depicted in the following examples show the use of the composition of this invention in exemplary toiletry and cosmetic applications. In the following examples, the names for each ingredient other than the composition of the invention are the CTFA (Cosmetics, Toiletry and Fragrance Association, Inc.) names.

### EXAMPLE 5

The following formulation illustrates the use of the invention that results in a hair conditioner that provides a deep conditioning effect.

Parts by weight

Part A

| | |
|---|---|
| Deionized Water | 79.40 |
| Butylene Glycol | 3.00 |
| Methylparaben | 0.20 |
| Propylparaben | 0.10 |

Part B

| | |
|---|---|
| Polyesteramine Type 6 | 5.00 |
| Cetearyl Alcohol (and) Ceteareth-20 | 4.00 |
| Trimethylolpropane Tricaprylate/Tricaprate | 8.00 |
| Tocopheryl Acetate | 0.30 |

The above components are formed into the composition by first combining the deionized water, butylenes glycol, methylparaben, and propylparaben, and warming to about 70° C. to about 75° C. with agitation. In a second vessel, the polyesteramine Type 6, cetearyl alcohol (and) ceteareth-20, trimethylolpropane tricaprylate/tricaprate, and tocopheryl acetate are combined and heated to about 70° C. to about 75° C. with agitation. The components of one vessel are then added to the other and are agitated until a uniform dispersion is obtained. The mixture is then allowed to cool to about 30 to about 35° C. and poured off to containers.

### EXAMPLE 6

The following formulation illustrates the use of the invention that results in a body wash that both cleanses and conditions the skin.

Parts by weight

Part A

| | |
|---|---|
| Deionized Water | 41.65 |
| Methylparaben | 0.20 |
| Propylparaben | 0.10 |
| Tetrasodium EDTA | 0.10 |

Part B

| | |
|---|---|
| Sodium Lauryl Sulfate | 19.00 |
| TEA-lauryl Sulfate | 12.00 |
| Cocamidopropyl Betaine (and) Glycerin | 14.00 |
| Polyesteramine Type 6 | 5.00 |

Part C

| | |
|---|---|
| Ethoxylated Coconut Oil | 5.00 |
| Tocopheryl Acetate | 0.20 |

Part D

| | |
|---|---|
| Citric acid | 2.75 |

The above-listed components are formed into the composition by the following procedure. Deionized water, methylparaben, propylparaben, and tetrasodium EDTA are added to a vessel and warmed with agitation to about 70° C. to about 75° C. until a uniform mixture is obtained. The polyesteramine Type 6, sodium lauryl sulfate, TEA lauryl sulfate, and cocamidopropyl betaine (and) glycerin are then added one by one. In a separate vessel, the ethoxylated coconut oil and tocopheryl acetate are mixed together and warmed to about 20° C. to about 25° C. The components of the second vessel are then added to the first and the mixture is allowed to cool to about 30° C. to about 35° C. The pH of the mixture is then adjusted to from about 6.0 to about 7.0 with citric acid. The mixture is then allowed to cool to room temperature and is poured off to containers.

### EXAMPLE 7

The following formulation illustrates the use of the invention that results in a shaving preparation lotion that leaves the face soft, supple, and provides a feeling of smoothness.

Parts by weight

Part A

| | |
|---|---|
| Stearic Acid | 20.00 |
| Pentaerythrityl Tetra C5-C9 Acid Esters | 10.00 |
| Glyceryl Stearate (and) PEG-100 Stearate | 1.75 |
| Polyesteramine Type 6 | 4.00 |

Part B

| | |
|---|---|
| Deionized Water | 57.45 |
| Glycerin | 5.00 |

Part C

| | |
|---|---|
| Triethanolamine | 0.80 |

Part D

| | |
|---|---|
| Propylene Glycol/Diazolidinyl | 1.00 |
| Urea/Metylparaben/propylparaben | |

The components listed above are formed into the composition by the following procedure. The stearic acid, pentaerythrityl tetra C5-C9 acid esters, glyceryl stearate (and) PEG-100 stearate, and polyesteramine Type 6 are added to a vessel and warmed with agitation to about 80° C. to about 85° C. until a uniform mixture is obtained. The deionized water and glycerin are added to a separate vessel and warmed with agitation to about 80° C. to about 85° C. until a uniform mixture is obtained. The components of the second vessel are then added to the first and the mixture is allowed to cool to about 70° C. to about 75° C. The TEA is then added and the mixture is then allowed to cool to about 40° C. to about 45° C. The propylene glycol/diazolidinyl urea/metylparaben/propylparaben are then added and the mixture is allowed to cool to about 30° C. to about 35° C. Mixing is then stopped, and the mixture is poured off to containers.

### EXAMPLE 8

Various prototype polyesteramines were tested for lubrication properties using the Falex Pin on Vee Block test procedure for determining the EP characteristics. The method is described in detail in the Annual Book of ASTM (American Society of Testing and Materials) Standards under method number ASTM-3233 which is incorporated herein by reference. The test specimen configuration consists of two stationary vee blocks made from AISI C-1137 steel clamped or loaded against a pin made from AISI 3135 steel that is capable of rotation. This produces a four-line contact configuration. The load can be varied and is applied directly to the rotating pin by means of the vee blocks using a ratcheting mechanism. The pin and vee blocks are submerged in the lubricant during testing. Loading the vee blocks against the rotating pin produces a torque that is modified by the lubricant.

In metalworking fluid (MWF) applications, such as cutting fluids, the lubricant is applied as an emulsion in water. In the test, the polyesteramine prototypes were made into a water dilutable metalworking fluid by merely dispersing them at a concentration of about 2% by weight. No emulsifiers were required since the polyesteramine prototypes all exhibited self-emulsifying behavior. In the test, the dispersion is heated to 51.7° C. and the pin is rotated against the vee blocks at 290 rpm at a load of 300 lbs. The load is then increased until the lubricant fails to maintain the load and either the locking pin that attaches the pin specimen breaks which indicates a weld failure, or the load cannot be maintained no matter how far the ratcheting mechanism is advanced which indicates a wear failure. Table 6 below summarizes the results obtained.

TABLE 6
Lubrication Properties of polyesteramine prototypes using Falex Pin on Vee
Method, ASTM D-3233.

| | Water | S 1500 | S 1750 | O 1250 | O 1500 | O 1700 | C 1450 | C 1600 |
|---|---|---|---|---|---|---|---|---|
| Failure | * | 2250 | 2000 | 2750 | >3000 | 2500 | 2250 | 2500 |
| Load, lbs. | | | | | | | | |
| Surface | * | Normal | Normal | Normal | Excellent | Normal | Normal | Normal |
| Finish | | | | | | | | |

Data for the control sample water was unobtainable because high friction caused the locking pin to break on initial loading of 300 lbs.

The data above indicates that all of the prototype polyesteramines have excellent lubrication properties in that a failure load of at least 2000 pounds indicates that a lubricant provides EP properties. Also, the surface finish on the pin when using polyesteramine O 1500 was very clean and bright which indicates that this lubricant is an excellent candidate for metal cutting operations.

### EXAMPLE 9

Polyesteramine O 1500 was evaluated as an aluminum cutting fluid using a modification of ASTM method D 2670 which is incorporated herein by reference. In this test, the same apparatus and vee blocks described in Example 8 are employed, however the pin is constructed of 7075-T6 aluminum. In the test, the fluid is held at 750 lbs. for fifteen minutes load after a three minute run in period at 250 lbs. The ratcheting mechanism is advanced as necessary to maintain the load, and the teeth in the ratcheting mechanism required to maintain the load at 750 lbs. are counted. Over the course of the test, the torque developed and the temperature of the fluid is monitored at ten second intervals.

The polyesteramine O 1500 was diluted at a 20:1 ratio in tap water prior to running the test. A commercial aluminum cutting fluid, Hocut 795 B (Houghton International, Inc., Valley Forge, Pa., USA), was also tested for comparative purposes at the same dilution ratio. Table 7 shows the results obtained.

TABLE 7
Lubrication Properties of polyesteramine prototypes
using Falex Pin on Vee Method, ASTM D-2670,
modified to test aluminum cutting behavior.

| | | Average | Temp. |
|---|---|---|---|
| Sample | Tooth Wear | Torque(inch-lb.) | Rise(° C.) |
| Polyesteramine O 1500 | 276 | 18.7 | 54 |
| Hocut 795 B | 87 | 19.6 | 65 |

High tooth wear at low torque with low temperature rise indicates a highly efficient aluminum cutting fluid. In the example above, a simple water dilution of the inventive polyesteramine prototype outperformed the commercial fluid.

While illustrated and described above with reference to certain specific embodiments, the present invention is nevertheless not intended to be limited to the details shown. Rather, the present invention is directed to polyesteramines and methods of production and use, and various modifications may be made in the details within the scope and range of equivalents of the description and without departing from the spirit of the invention.

1. A polyesteramine comprising:
a tertiary amine group;
an ester linkage; and
an alkyl chain.

2. The polyesteramine of claim 1, further comprising a hydroxyl group.

3. The polyesteramine of claim 2, further comprising a carboxylic acid group.

4. The polyesteramine of claim 1, further comprising a carboxylic acid group.

5. The polyesteramine of claim 1, wherein the pH is between about 7.0 and about 10.0.

6. The polyesteramine of claim 1, wherein the molecular weight of the polyesteramine is between about 600 Daltons and about 5,000 Daltons.

7. The polyesteramine of claim 1, further comprising an aryl chain.

8. A polyesteramine comprising:
a tertiary amine group;
an ester linkage; and
an aryl chain.

9. The polyesteramine of claim 8, further comprising a hydroxyl group.

10. The polyesteramine of claim 9, further comprising a carboxylic acid group.

11. The polyesteramine of claim 8, further comprising a carboxylic acid group.

12. The polyesteramine of claim 8, wherein the pH is between about 7.0 and about 10.0.

13. The polyesteramine of claim 8, wherein the molecular weight of the polyesteramine is between about 600 Daltons and about 5,000 Daltons.

14. The polyesteramine of claim 8, further comprising an alkyl chain.

15. A polyesteramine having the formula: 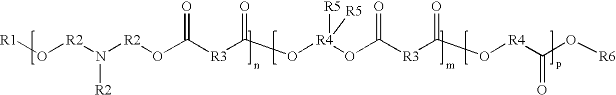
wherein:
the polymer is a random co-polymer;
R1 is H or R7(C═O), wherein R7 is C₅-C₃₆ aliphatic and/or C₆ aromatic;
R2 is C₁-C₆ aliphatic;
R3 is independently a divalent radical having from 0 to 34 carbon atoms, and/or a divalent aromatic radical having from 6 to 12 carbon atoms, and/or a divalent aromatic radical having 2 carboxylic acid groups;
R4 is independently C₁-C₂₀₀ aliphatic having 0-100 oxygen atoms as ether groups;
R5 is —H, —R4-OH, and/or 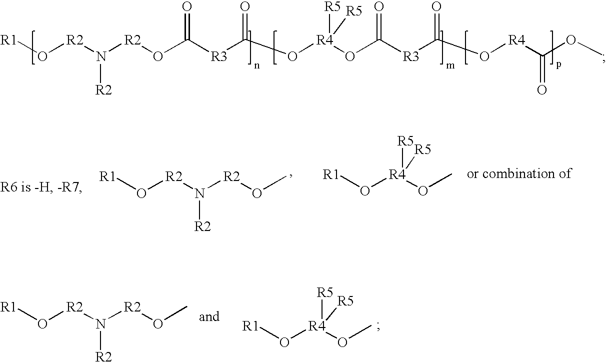
n is an integer between 1-50;
m is an integer between 0-50; and
p is an integer between 0-20.

16. The polyesteramine of claim 15, wherein R7 is linear, branched or a combination thereof.

17. The polyesteramine of claim 15, wherein the divalent radical of R3 is linear, branched or a combination thereof.

18. The polyesteramine of claim 15, wherein the molecular weight of the polyesteramine is between about 600 Daltons and about 5,000 Daltons.

19. A method of producing a polyesteramine comprising reacting:
a tertiary amine functional polyol;
a polyfunctional carboxylic acid; and
at least one member selected from the group consisting of monofunctional carboxylic acids and monofunctional alcohols.

20. The method of claim 19, further comprising reacting at least one member selected from the group consisting of alcohol, polyol and hydroxyacid.

21. The method of claim 19, wherein the tertiary amine functional polyol comprises methyldiethanolamine.

22. The method of claim 19, wherein the polyfunctional carboxylic acid comprises at least one member from the group consisting of adipic acid, cyclohexanedicarboxylic acid, sebacic acid, azelaic acid, dodecanedioic acid, phthalic acid, isophthalic acid, terephthalic acid, trimellitic acid, dimer acid, trimer acid, 2,6-naphthalene dicarboxylic acid, and pyromellitic acid.

23. The method of claim 19, wherein the monofunctional carboxylic acid comprises at least one member from the group consisting of benzoic acid, 2-ethylhexanoic acid, isononanoic acid, lauric acid (C-12), myristic acid (C-14), palmitic acid (C-16), isomyristic acid (Iso C-14), isopalmitic acid (Iso C-16), isostearic acid (Iso C-18), coconut fatty acid (C8-C18), oleic acid (C18:1), and behenic acid (C-22).

24. The method of claim 19, wherein the monofunctional alcohol comprises at least one member from the group consisting of tridecyl alcohol, Guerbet alcohols, coconut fatty alcohols, isooleic alcohol, and isostearyl alcohol.

25. The method of claim 20, wherein the polyol comprises at least one member from the group consisting of propylene glycol, 1,3-butylene glycol, cyclohexanedimethanol, trimethylpentanediol, polyoxyalkylene glycol, butyl ethyl propanediol, dipropylene glycol, neopentyl glycol, glycerol, trimethylolpropane, pentaerythritol, and dipentaerythritol.

26. The method of claim 20, wherein the hydroxy acid comprises at least one member from the group consisting of lactic acid, glycolic acid, hydroxystearic acid, and citric acid.

27. The method of claim 19, wherein the polyesteramine has an acid value from 0 to about 100 mg KOH/g.

28. The method of claim 19, wherein the polyesteramine has an acid value from 0 to about 50 mg KOH/g.

29. A lubricant composition comprising the polyesteramine of claim 1.

30. A cosmetic composition comprising the polyesteramine of claim 1

31. A method of using a polyesteramine comprising applying the polyesteramine of claim 1 to skin, hair, nails, keratinous fibers, semimucous membranes and/or mucous membranes.

32. A method of using a polyesteramine for industrial lubricant applications comprising applying the polyesteramine of claim 1 to a surface, wherein the polyesteramine is an emulsion in water.

33. A lubricant composition comprising the polyesteramine of claim 8.

34. A cosmetic composition comprising the polyesteramine of claim 8.

35. A method of using a polyesteramine comprising applying the polyesteramine of claim 8 to skin, hair, nails, keratinous fibers, semimucous membranes and/or mucous membranes.

36. A method of using a polyesteramine for industrial lubricant applications comprising applying the polyesteramine of claim 8 to a surface, wherein the polyesteramine is an emulsion in water.

37. A composition produced by reacting at least one tertiary amine functional polyol, at least one polyfunctional carboxylic acid and at least one member selected from the group consisting of monofunctional acids and monofunctional alcohols.

38. The composition of claim 37, further comprising reacting at least one member selected from the group consisting of alcohol, polyol and hydroxyacid.

39. The composition of claim 37, wherein the tertiary amine functional polyol comprises methyldiethanolamine.

40. The composition of claim 37, wherein the polyfunctional carboxylic acid comprises at least one member from the group consisting of adipic acid, cyclohexanedicarboxylic acid, sebacic acid, azelaic acid, dodecanedioic acid, phthalic acid, isophthalic acid, terephthalic acid, trimellitic acid, dimer acid, trimer acid, 2,6-naphthalene dicarboxylic acid, and pyromellitic acid.

41. The composition of claim 37, wherein the monofunctional carboxylic acid comprises at least one member from the group consisting of benzoic acid, 2-ethylhexanoic acid, isononanoic acid, lauric acid (C-12), myristic acid (C-14), palmitic acid (C-16), isomyristic acid (Iso C-14), isopalmitic acid (Iso C-16), isostearic acid (Iso C-18), coconut fatty acid (C8-C18), oleic acid (C18:1), and behenic acid (C-22).

42. The composition of claim 37, wherein the monofunctional alcohol comprises at least one member from the group consisting of tridecyl alcohol, Guerbet alcohols, coconut fatty alcohols, isooleic alcohol, and isostearyl alcohol.

43. The composition of claim 38, wherein the polyol comprises at least one member from the group consisting of propylene glycol, 1,3-butylene glycol, cyclohexanedimethanol, trimethylpentanediol, polyoxyalkylene glycol, butyl ethyl propanediol, dipropylene glycol, neopentyl glycol, glycerol, trimethylolpropane, pentaerythritol, and dipentaerythritol.

44. The composition of claim 38, wherein the hydroxy acid comprises at least one member from the group consisting of lactic acid, glycolic acid, hydroxystearic acid and citric acid.

45. The composition of claim 37, wherein the composition has an acid value from 0 to about 100 mg KOH/g.

46. The composition of claim 37, wherein the composition has an acid value from 0 to about 50 mg KOH/g.

47. A hair conditioner produced by mixing deionized water, butylene glycol, methylparaben, propylparaben, the polyesteramine of claim 1, cetearyl alcohol (and) ceteareth-20, trimethylolpropane tricaprylate/tricaprate and tocopheryl acetate.

48. A body wash produced by mixing deionized water, methylparaben, propylparaben, tetrasodium EDTA, sodium lauryl sulfate, TEA-lauryl sulfate, cocamidopropyl betaine (and) glycerin, the polyesteramine of claim 1, ethoxylated coconut oil, tocopheryl acetate and citric acid.

49. A shaving preparation lotion produced by mixing stearic acid, pentaerythrityl tetra C5-C9 acid esters, glyceryl stearate (and) PEG-100 stearate, the polyesteramine of claim 1, deionized water, glycerin, triethanolamine and propylene glycol/diazolidinyl urea/methylparaben/propylparaben.
